(19) United States
(12) Patent Application Publication
(10) Pub. No.: US 2021/0285633 A1
(43) Pub. Date: Sep. 16, 2021

(54) WEARABLE UV LIGHT DEVICES

(71) Applicant: Arash Esmailzadeh, (US)

(72) Inventor: Arash Esmailzadeh, Los Angeles, CA (US)

(21) Appl. No.: 17/200,587

(22) Filed: Mar. 12, 2021

Related U.S. Application Data
(60) Provisional application No. 62/988,767, filed on Mar. 12, 2020.

(51) Int. Cl. F21V 33/00; A61L 9/20; F21V 23/04

(52) U.S. Cl. CPC F21V 33/0008; A61L 9/20; F21Y 2115/10; F21V 23/0478; F21V 23/045

(57) ABSTRACT

A wearable ultraviolet (UV) light device may include one or more UV light-emitting devices, such as LEDs, and a housing that is attachable to a wearable article of clothing. When the housing is attached to the wearable article of clothing, the one or more UV light-emitting devices may be positioned and configured to emit light onto or in front of a face of a user disinfect air passing through the light in front of the face of the user. The wearable article of clothing may include hats, headbands, neckband, T-shirts, etc. The device may also include a processor that automatically activates the device in response to images captured by a camera, motion detection, sounds or voice commands, wireless commands from a smart phone, locations detected by a GPS device, and so forth.

### CROSS-REFERENCES TO RELATED APPLICATIONS

This application claims the benefit of U.S. Provisional Patent Application No. 62/988,767 filed on Mar. 12, 2020, which is incorporated herein by reference.

### BACKGROUND

Ultraviolet (UV) light is defined as electromagnetic radiation having a wavelength greater than approximately 10 nm and less than approximately 400 nm, which generally falls between the visible light spectrum and the x-ray spectrum. The most common form of UV light experienced by most people comes from the output of the sun. Other sources include special UV lights, tanning lamps, black lights, incandescent lamps, gas-discharge lamps, ultraviolet LEDs, ultraviolet lasers, and other artificial sources. The lower wavelength limit of vision in most humans is greater than the 400 nm range of UV light, so UV rays are typically invisible to humans. Therefore, despite the prevalence of UV light in every daylight, most humans operate unaware of its presence.

UV light has many practical applications. Besides the beneficial effect of generating vitamin D in humans with limited exposure, UV light has often been used for sterilization and disinfection of surfaces. For example, UV lamps have been used to sterilize workbenches and tools in biology labs and medical facilities. Specifically, UV radiation at around 250 nm may have a very effective germicidal effect of damaging the RNA/DNA of microorganisms, thus rendering reproduction impossible to prevent germ spread. UV radiation is also commonly used in wastewater treatment and the disinfection of drinking water. Some food processes also use UV radiation to kill unwanted microorganisms. Despite these uses for sterilizing surfaces, UV light has not been used to prevent the spread of airborne diseases in humans. Therefore, improvements in the art are needed.

### BRIEF SUMMARY

In some embodiments, a wearable ultraviolet (UV) light device may include one or more UV light-emitting devices, and a housing that is attachable to a wearable article of clothing. When the housing is attached to the wearable article of clothing, the one or more UV light-emitting devices may be positioned and configured to emit light onto or in front of a face of a user.

In any embodiments, any and/or all of the following features may be implemented in any combination and without limitation. The device may include means for attaching the housing to the wearable article of clothing. The means for attaching the housing to the wearable article of clothing may allow the device to be removable from the wearable article of clothing. The one or more light-emitting devices may be configured to emit far-UV light. The one or more light-emitting devices may include a plurality of UV light-emitting diodes (LEDs). The device may include the wearable article of clothing. The wearable article of clothing may include a hat, and the housing may be attached to a bottom side of a brim of the hat. The wearable article of clothing may include a headband or neckband. The housing may be embedded within the wearable article of clothing. The device may include a camera and one or more processors, where the camera may be configured to capture an image, and the one or more processors may be programmed to determine whether to activate the one or more UV light-emitting devices based at least in part on the image. The processor may be programmed to analyze the image and activate the one or more UV light-emitting devices when the image includes a human approaching the camera. The camera may include a thermal camera, and the processor may be programmed to analyze the image and activate the one or more UV light-emitting devices when the image includes a human with a fever temperature. The device may include a wireless communication chip configured to communicate with a smart phone, and one or more processors programmed to activate the one or more UV light-emitting devices in response to a command received from the smart phone through the wireless communication chip. The device may include a motion sensor and one or more processors programmed to activate the one or more UV light-emitting devices in response to motion detected by the motion sensor. The device may include a microphone and one or more processors programmed to activate the one or more UV light-emitting devices in response to a voice detected by the microphone. The one or more processors may be programmed to determine whether the voice detected by the microphone belongs to the user. The device may include a power supply and an on/off switch. The one or more light-emitting devices may be positioned on opposite sides of the face of the user. The device may include a global positioning system (GPS) sensor and one or more processors, where the one or more processors may be programmed to activate the one or more UV light-emitting devices in response to entering a location detected by the GPS sensor. The device may include one or more processors programmed to activate the one or more UV light-emitting devices in response to detecting another nearby wearable UV light device.

### BRIEF DESCRIPTION OF THE DRAWINGS

A further understanding of the nature and advantages of various embodiments may be realized by reference to the remaining portions of the specification and the drawings, wherein like reference numerals are used throughout the several drawings to refer to similar components. In some instances, a sub-label is associated with a reference numeral to denote one of multiple similar components. When reference is made to a reference numeral without specification to an existing sub-label, it is intended to refer to all such multiple similar components.

FIG. 1 illustrates a wearable UV light device according to some embodiments.

FIG. 2 illustrates a UV light device that may be an integrated part of a wearable item of clothing, according to some embodiments.

FIG. 3 illustrates how a UV light device may be positioned or angled to control the direction of the light, according to some embodiments.

FIG. 4 illustrates an example of a light device that is attached to a headband, according to some embodiments.

FIG. 5 illustrates a light device that is affixed to a wearable item on the body of the user, according to some embodiments.

FIG. 6 illustrates a simplified block diagram of a wearable UV device, according to some embodiments.

### DETAILED DESCRIPTION

Described herein are embodiments for wearable devices that emit safe levels of ultraviolet (UV) light in front of the facial region of a user. Seasonal illnesses, such as colds and flus, tend to spread quickly from person-to-person when the bacteria or viruses are airborne. An infected person may cough or touch their hands and thereby spread the germs to common surfaces or into the air. Uninfected persons may then touch the infected surfaces and/or breathe in the air containing the germs. In either case, the germs typically enter an uninfected person by way of their face, either through direct inhalation or through hand contact. Many different disinfectant solutions have been tried, but none are able to form a reliable barrier to keep these germs out of user's face.

Broad-spectrum ultraviolet (UV) light kills bacteria and viruses, and it is currently used to decontaminate surgical equipment in some circumstances. The embodiments described herein use a safe version of the UV light spectrum to create a virtual barrier in front of a user's face to prevent the spread of bacteria or viruses. The embodiments described herein can protect against any type of germ, including viruses, bacteria, and/or other chemicals or substances that may be harmful. Collectively, these different threats are referred to as “contaminants.”

They embodiments discussed herein use UV light as a non-limiting example. Whenever UV light is discussed in this disclosure, any light source associated with any other portion of the light spectrum may be substituted in its place in any combination and without restriction. For example, some embodiments may use a narrow spectrum of UV light referred to as “far-UV” as a safe option. Far-UV light is effective for killing bacteria such as Methicillin-resistant S. Aureus (MRSA) bacteria, while at the same time being relatively safe for human contact. Far-UV light has a very limited range and typically does not penetrate through the outer layer of human skin or the tear layer of the human eye. Because most contaminants are much smaller than human skin cells, the far-UV light can still be very effective at neutralizing contaminants (e.g., reaching bacterial DNA to kill the bacteria) without posing a safety risk.

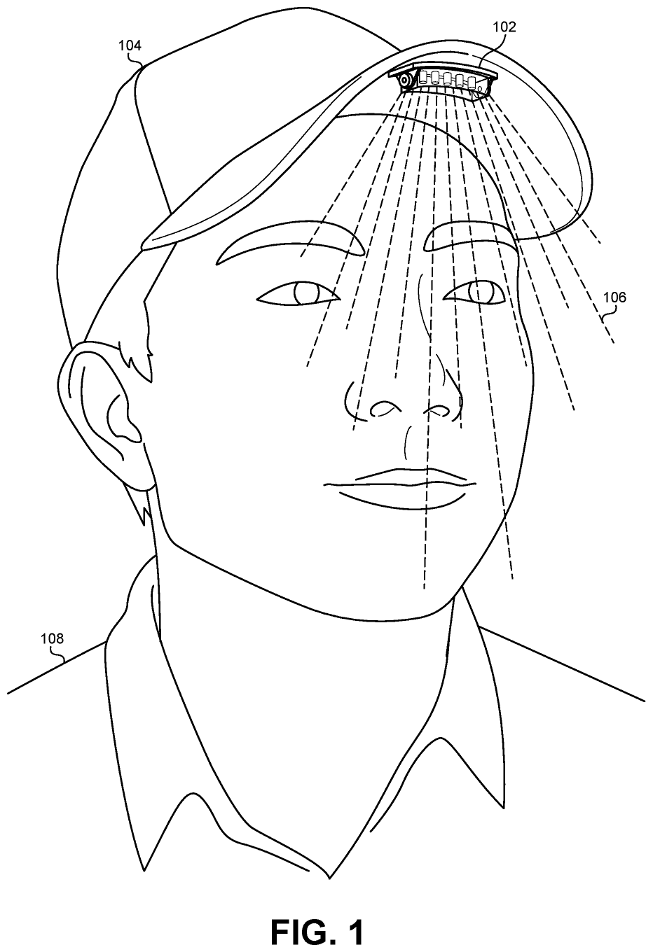

FIG. 1 illustrates a wearable UV light device 102 according to some embodiments. In this example, a user 108 may wear a piece of headwear, such as a hat 104. The light device 102 may be attached to the brim of the hat 104. The light device 102 may be attached with any suitable type of adhesive or attachment mechanism. For example, an adhesive sticker may be placed on the back of the light device 102 and used to affix the light device 102 to the brim of the hat 104. Other embodiments may use screws, rivets, or other devices to more permanently affixed the light device 102 to the brim of the hat 104.

The UV light device 102 may include one or more light-emitting devices, such as UV bulbs or light emitting diodes (LEDs). The bulbs or LEDs may be configured to emit light in the desired UV spectrum. In some embodiments, filters or lenses may be placed in front of the LEDs or bulbs to only allow the desired UV spectrum to be emitted out of the device 102. The device 102 may include an on/off switch or button along with a power supply to power the light-emitting devices. For example, the device may use a AAA battery, a “button” battery (e.g., CR2032, CR2016, etc.), a rechargeable lithium ion battery, a rechargeable capacitor, and/or the like.

The UV light device 102 may be angled such that it emits light 106 in front of the face of the user 108. For example, the device 102 may include a row of bulbs or LEDs that emit light in a plane in front of the face of the user 108. This may form a virtual barrier through which contaminants will pass before reaching the face of the user 108. In some embodiments, the angle of the device 102 may be configured such that the light 106 does not touch the face of the user 108, but instead is projected down and away from the face of the user 108. As contaminants pass through the light 106 they may be neutralized before they reach the face of the user 108. For example, if the user 108 touches a contaminated surface, then moves to touch their face, the light 106 may neutralize the germs on the hand of the user 108 before they reach the mouth/eyes of the user 108. In another example, if a contaminated person coughs in the vicinity of the user 108, the airborne contaminants may be neutralized by the light 106 before they would be breathed in by the user 108.

The UV light device 102 may include a housing as depicted in FIG. 1. The housing may include a plastic housing that encompasses electronics, such as a battery, a processor, an LED driver circuit, a switching circuit, and/or the like. In some embodiments, the bulbs or LEDs that emit light in front of or onto the face of the user may be exposed through or protrude from the housing. The housing may be attachable to a wearable article of clothing. The article of clothing may include a hat, a headband, a neckband, a T-shirt, sweatshirt, and so forth. The housing may include means for attaching to the article of clothing, such as Velcro, a sticker, adhesive, screws, clips, clasps, tape, holes or surfaces which may be sewn into the clothing, and/or other equivalents. For example, the housing may include Velcro that attaches to a brim of a hat as illustrated in FIG. 1. In some embodiments, the housing may be permanently affixed to the wearable article of clothing, and the means for attachment may include protrusions or flanges that are sewn into the clothing. In an example described below, the housing may be attached to a wearable article of clothing by being embedded within the clothing, such as within the brim of the hat or within a neckband or headband.

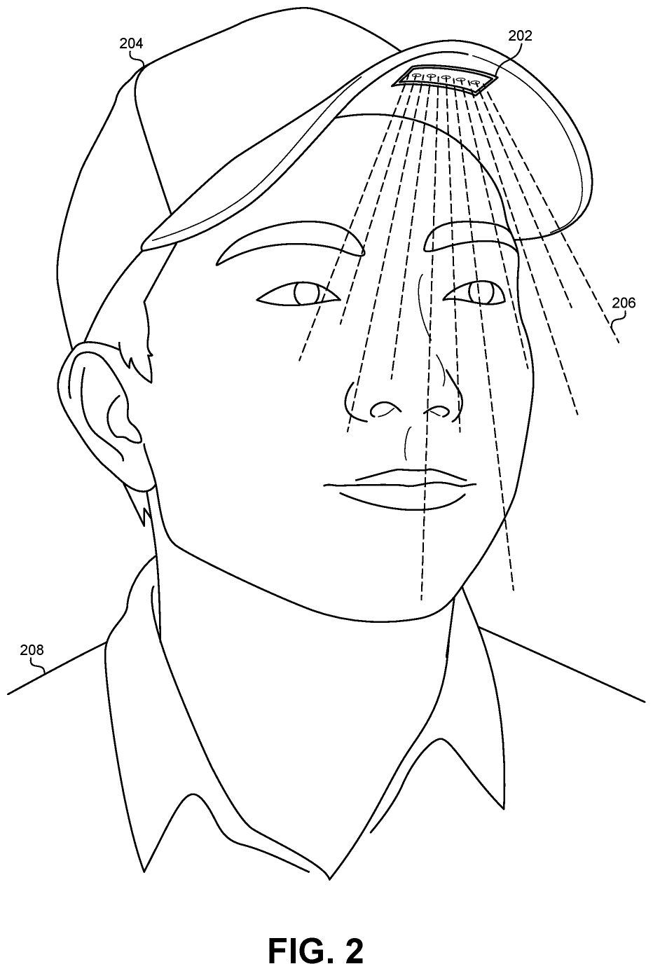

FIG. 2 illustrates a UV light device 202 that may be an integrated part of a wearable item of clothing, according to some embodiments. This example is similar to the example of FIG. 1, except the light device 202 is integrated as part of the wearable item, such as a hat 204. Instead of using a separate electronic device that can be fitted onto the hat 204, the light device 202 may be integrated into the brim of the hat 204. The light device 202 may include a thin strip of LEDs or other light-emitting devices that can be embedded in the brim. The thin strip of LEDs may be flexible such that it can flex with the brim of the hat 204. The thin strip of LEDs may also be sized such that the entire strip fits substantially within the thickness of the brim of the hat 204. In some embodiments, the LED flashlight strip may protrude somewhat from the bottom side of the brim of the hat 204 (e.g., 1 mm, 2 mm, 3 mm, etc.).

The controls and/or battery described above may be wired to the light device 202 and located elsewhere in the hat 204. For example, the battery pack or on/off control may be located inside the hat 204, located on the back of the hat 204, or wired to a separate control device that can be held by the user, placed in their pocket, or otherwise located away from the brim of the hat 204.

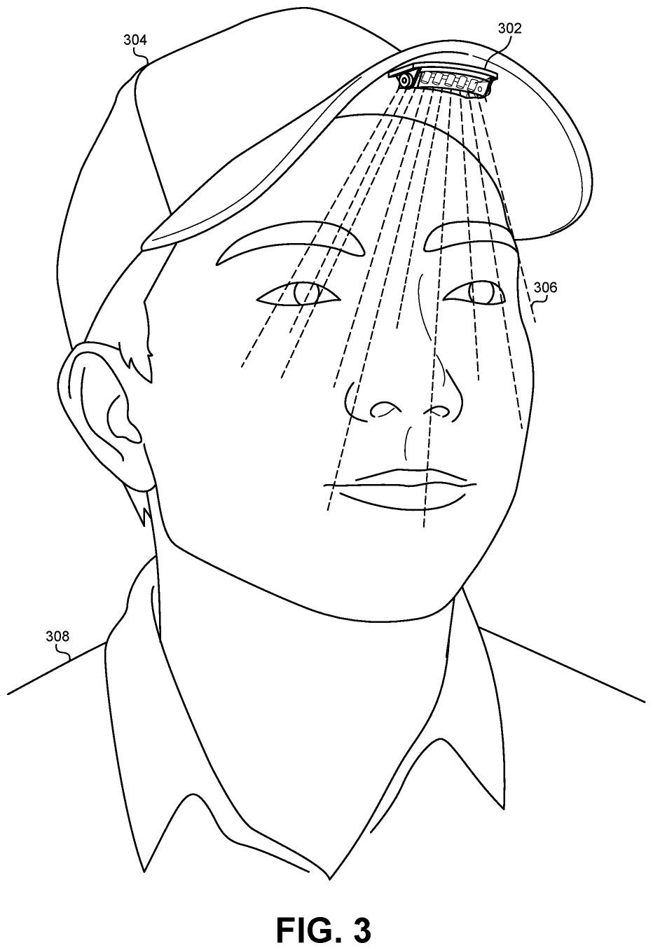

FIG. 3 illustrates how a UV light device 302 may be positioned or angled to control the direction of the light 306, according to some embodiments. In contrast to FIG. 1, this example of a light device 302 allows the user to change the angle of the light sources (e.g., LEDs, UV bulbs, etc.) to project the light 306 at different angles. For example, this user has directed the light 306 such that the light begins to shine on the user's face just above the nostrils and ends just below the mouth. This has the effect of shining light directly on the orifices through which contaminants commonly enter. As described above, safe UV light spectrums may be used when the light 306 is directed to the face of the user 308.

In some embodiments, the angle or orientation of the light 306 emitted from the light device 302 may be determined by the physical components. For example, the device 302 may include LEDs that are oriented at an angle such that when the device 302 is affixed to the brim of the hat 304, the light 306 shines on to and/or in front of the face of the user 308 as desired. Some embodiments may allow the user 308 to change the orientation of the LEDs/bulbs of the device 302 or the orientation of the device itself as the device 302 is worn. For example, a knob on the side of the device 302 may be used to rotate the orientation or angle of the LEDs/bulbs to change the direction of the light 306. In some embodiments, a filter or window on the device through which the light 306 exits the device may be rotated or moved. For example, a rectangular opening on the bottom of the device 302 may be rotated to allow the light 306 to exit the device 302 at different angles.

As described above, the UV light spectrum is used here is an example, but is not meant to be limiting. Some embodiments may replace the UV light source with a laser light source . The laser light source may then be aimed directly at the nostrils and/or mouth of the user to neutralize any contaminants that would enter these orifices. Some embodiments may include safety glasses that may be worn by the user 308 to prevent laser light from entering their eyes.

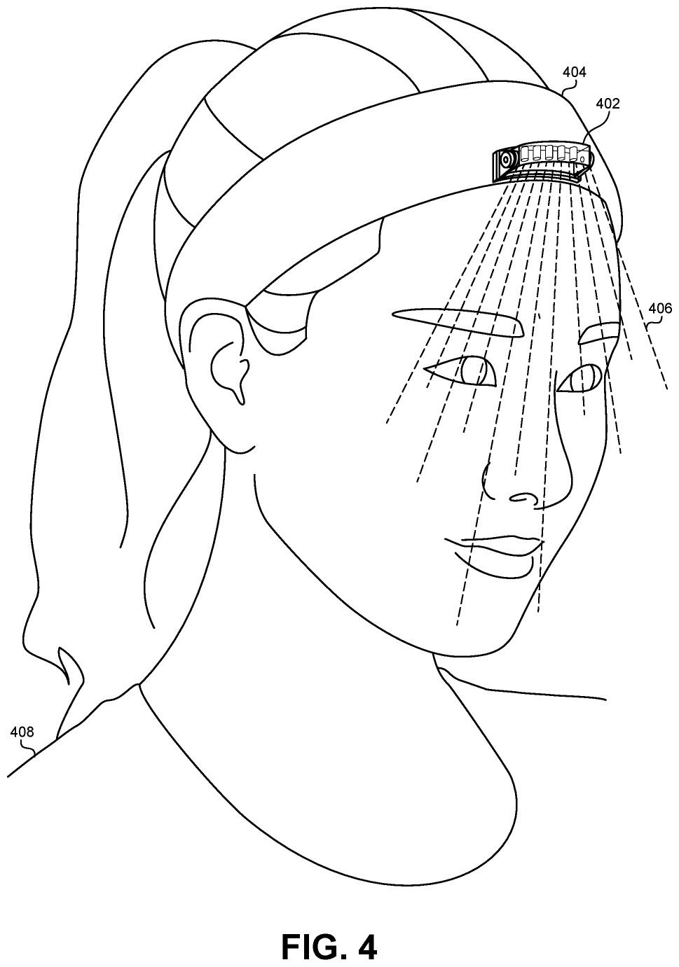

In the example described above, the light device 102, 202, 302 was embedded or affixed to a hat. The hat is used as an example, but is not meant to be limiting. The light device may be embedded or affixed to any wearable item such that the light emitted by the device is projected in front of or onto the face of the user. FIG. 4 illustrates an example of a light device 402 that is attached to a headband 404, according to some embodiments. Instead of affixing the light device 402 to the brim of the hat, these embodiments affix the light device 402 to the front of a headband 404. The light device 402 may be a permanent part of the headband 404. The light device 402 may also be attached to the headband 404 by the user 408 by any adhesive or mounting technology (e.g., glue, sticker, tape, adhesive, screws, hook-and-loop, and/or the like). As described above, the device 402 may include a battery, a control device, and/or other electronics. The battery and/or control device may be an integrated part of the device 402 and/or may be routed to different parts of the item of clothing. For example, the battery and/or control of the device may be located on the back of the headband 404 behind the hair of the user 408 to balance the weight of the headband 404 and/or conceal the battery compartment. Also, the angle and orientation of the LEDs/bulbs in the light device 402 may be changeable such that the angle of the light 406 may be both emitted in front of the face of the user 408 and/or onto the face of the user as described above.

The hat and the headband described above are examples of many different wearable items that to which the light devices may be affixed. Other wearable items that are compatible with these light devices may include sunglasses, prescription glasses, headphones, snow caps, hearing aids, bandannas, sweat bands, and/or any other wearable item. Additionally, some embodiments can be affixed directly to the user. For example, the device 402 may be provided with an adhesive sticker on the backside of the device 402 that can be affixed directly to the skin of the user 408. For example, the user may enter an area believed to include contaminated individuals, and the user 408 may “stick” the device 402 to their forehead, cheek, chest, etc., to provide temporary but immediate protection. Some embodiments may also provide integrated elements that allow the user to wear the device 402 without a separate wearable item. For example, the device 402 may be included on a strap or plastic extension that is hung from the ears of the user 408 such that light sources are angled in front of the nose/mouth of the user 408. A headset may be worn such that a device is oriented in front of the user's mouth/nose area (e.g., similar to an audio headset with a microphone).

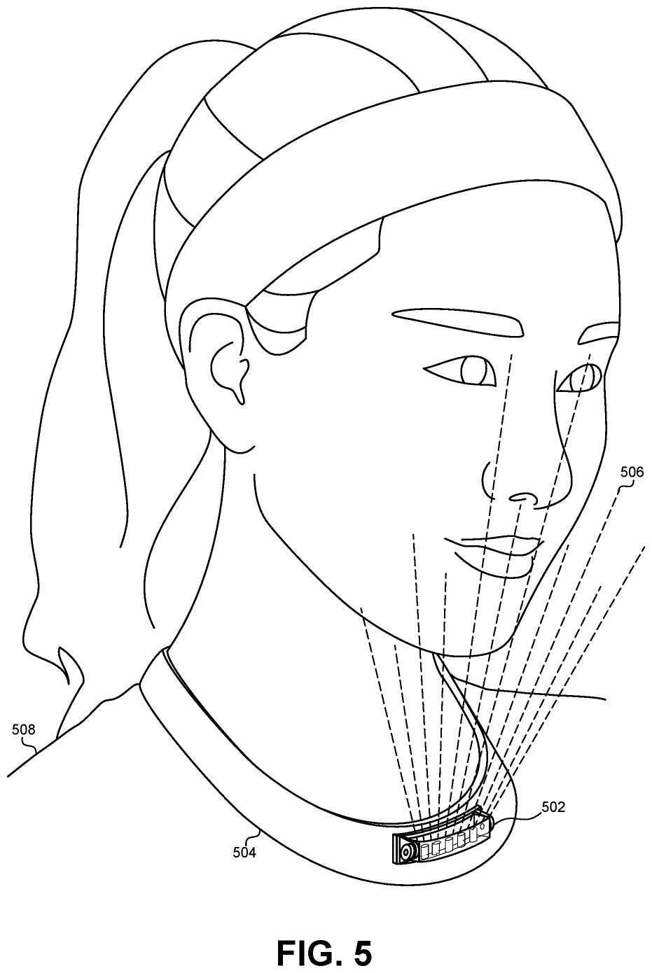

FIG. 5 illustrates a light device 502 that is affixed to a wearable item on the body of the user 508, according to some embodiments. In this example, the light device 502 may be attached to a necklace or neckband 504 that is worn around the neck of the user 508. The light 506 may then be emitted upwards in front of the face of the user 508 or onto the face of the user 508 as described above. This light device 502 may function in a manner similar to the light devices described above that were mounted on wearable items above the face of the user 508, except that the light 506 may be emitted in an opposite direction. Note that the neckband 504 may be a removable, wearable item that is separate from other wearable items. Alternatively, the neckband 504 may be part of a T-shirt or necklace worn by the user.

In some embodiments, the light device 502 may be affixed to, or part of a shirt worn by the user 508. For example, the light device 502 may be mounted to a collar or front of a shirt. The light device 502 may also be mounted to a shoulder area of the user to project the light 506 in front of the face of the user. The light device 502 may also be affixed to, or part of other wearable items, such as neckties, cummerbunds, suit coats, scarves, and/or any other wearable item that may allow the light 506 to be projected in front of, or onto the face of the user 508.

Some embodiments may include multiple light devices 502. For example, the user may wear a configuration that includes a first light device that is affixed to a brim of the hat, as well as a second light device that is affixed to a neckband 504 or T-shirt. In another example, a user may wear a configuration that includes two light devices that are mounted to booms or microphone-type extensions that emit light on either side of the face of the user. In another example, a user may wear light devices on each shoulder to shine in front of their face. In another example, a user may wear a mask or other facial covering that includes a ring of light devices that surround the face of the user. For example, a hazmat suit may include a window that may be lined with a string of light devices that shine across the window in front of the face of the user. Other masks similar to a “welder” mask or/guard may be worn that include light devices that shine across the mask in front of the user's face. In configurations that use a plurality of light sources, each of the light sources may be controlled individually or together using a common power source and/or common control.

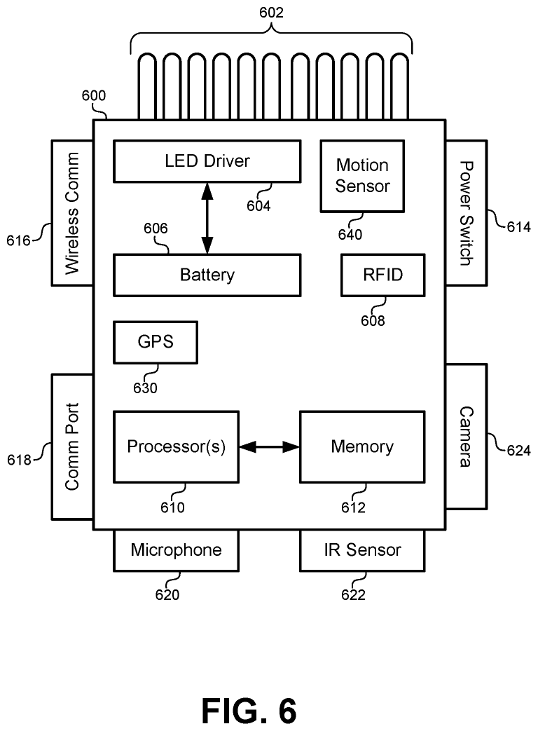

FIG. 6 illustrates a simplified block diagram of a wearable UV device 600, according to some embodiments. The device 600 may include a battery 606, such as a AAA battery, a rechargeable lithium-ion battery that may be charged through a communication port 618, and/or the like. The battery 606 may be coupled to an LED driver 604 configured to receive voltage/current from the battery 606 and provide driver signals to one or more LEDs 602. The LED driver 604 and/or the battery 606 may be controlled with a power switch 614 that is accessible through a housing of the device 600. For example, a user may toggle the power switch 614 to activate the one or more LEDs 602.

In some embodiments, the control of the device 600 may be moved away from the light device itself for the convenience of the user. For example, some embodiments may also include a Bluetooth or Wi-Fi communication chip and/or antenna 616 and a microprocessor 610 or microcontroller that allows the device to communicate with an external control device. Some embodiments may allow the light device to communicate with an app operating on a smart phone, a PDA, a tablet computer, a desktop computer, a smart watch, and/or any other personal computing device. For example, the user may turn on/off the light using controls on a smart phone app. The user may also change the angle and/or intensity of the light using controls on their smart phone app.

Some embodiments may also allow the device 600 to communicate with other similar devices that are nearby. For example, some embodiments may include a radio frequency identification (RFID) tag 608 that can be detected by other nearby units. For example, the device 600 may use the wireless communication chip and/or antenna 616 to detect the presence of RFID tags on other nearby units. When nearby units are detected, the processor 610 may cause the one or more LEDs 602 to be activated automatically. For example, when a user approaches another user, both of which use wearable UV devices 600, the devices may automatically be alerted to the presence of the other device and activate the LEDs 602.

Some embodiments may also include a thermal camera and/or a visible light camera 624 that is integrated with the device 600. These cameras 624 may be used to provide an additional analysis of the surrounding environment, to provide alerts, and/or to control the operation of the LEDs 602 themselves. For example, some embodiments may include a thermal camera that is angled outward in front of the face of the user. The thermal camera may be positioned such that it can capture images of the surrounding area, such as within the field of view of the user. In the example of FIG. 1, the thermal camera may be oriented such that it aims out from under the brim of the hat to capture a view of the surrounding area as would be seen by the user. A visible light camera may be used to detect the presence of other people, such that when the device 600 automatically activates the LEDs 602 when near other people. This same function may be carried out with the thermal camera, which may receive thermal signatures or images of nearby individuals. The processor 610 may compare these thermal signals to known baselines representing humans, and automatically activate the LEDs 602.

The thermal camera may be used to identify individuals with an elevated temperature. For example, many common flus or other illnesses may cause a fever or elevated temperature in a contaminated individual. When the thermal camera sees an image of a surrounding environment that includes an image of a contaminated individual, the microprocessor on the light device may execute known algorithms to estimate a temperature of the individual from the thermal image. This temperature can then be compared to thresholds that may indicate a dangerous level of temperature elevation. Alternatively, images from the thermal imager may be encoded, compressed, encrypted, and/or otherwise sent to another computing device, such as the user's smart phone. An app on the smart phone may then process the image to determine a temperature of the individual, If this processing indicates that an individual with an elevated temperature is near the user, the smart phone and/or device may generate an alarm or alert. For example, the smart phone may generate a push notification to the user indicating that a nearby individual has an elevated temperature. Vibrations, sounds, and/or lights may be generated by the light device and/or by the smart phone as an alert.

Some embodiments may automatically trigger activation of the light device 600 to begin emitting light in front of, or onto the face of the user based on conditions detected in the surrounding area. For example, when the thermal camera captures an image of a user with an elevated temperature, and the microprocessor 610 or smart phone may determine that the elevated temperature exceeds a predetermined threshold, and the light device may be activated automatically in response to begin projecting a protective barrier of light in front of or onto the face of the user. The light device 600 may remain activated as long as the individual with the elevated temperature remains in the visible range of the user. Some embodiment may also leave the light device activated for a time interval after the individual with the elevated temperature leaves (e.g., the device may stay on for 15 minutes after an individual with a fever leaves the area).

The light device may also be automatically activated when other individuals are detected in the vicinity of the user, regardless of their temperature. Some embodiments may use the thermal camera to detect a user that is nearby, even if they have a normal temperature. Thermal signatures from the thermal image may be used to distinguish between environmental heat sources (e.g., furnace vents, heaters, windows, etc.) and thermal signatures from human and/or animal users. The thermal signature may then be compared to a range of acceptable temperatures for humans and/or animals to detect their presence. When another human or animal comes within a predetermined range (e.g., 5 feet, 10 feet, 15 feet, 20 feet, 25 feet, 30 feet, 35 feet, 40 feet, and so forth), the light device may be automatically activated to begin emitting light in front of, or onto the face of the user to provide protection. Thermal signatures may also be obtained from a dedicated infrared (IR) sensor 622. For example, a passive IR sensor may detect motion from human -sized entities to activate the device 600.

In the embodiments described above, the one or more processors 610 may be programmed to analyze images were thermal images to detect human forms, human temperature signatures, motion, and other characteristics from the images. Standard computer vision algorithms may be used to detect humans within the images.

Some embodiments may include a motion sensor 640 that is configured to detect motion of the user and activate automatically in response. For example, the motion sensor 640 may include an accelerometer that detects when the user begins to move. The processor 610 may be programmed to activate the LEDs 602 when the user begins to move. For example, the device 600 may be off as the user sits at their desk, and turn on when the user stands to move throughout an office environment.

Other embodiments may include a microphone 620 that detects voices that are not attributed to the user. For example, when the microphone receives an unrecognized voice, the system may determine that another human is present in the area and automatically activate the light device in response. In some embodiments, the microphone 620 may also be used to perform voice recognition such that the light device can be controlled using voice commands. For example, the user can issue a verbal command, such as “activate” or “turn on the light.” This voice command may be received by the device and sent to a microprocessor 610, a smart phone, and/or a connected server through a Wi-Fi, router, or cellular connection to perform a known voice recognition algorithm and provide a command to the light device to activate. Additionally, the microphone 620 may listen for the voice of the user and automatically activate the LEDs 602 when the user begins speaking. Since germs are often projected while speaking, the device 600 can turn on to disinfect the air and/or projectile droplets that are emitted from the user's mouth. The microphone 602 may also listen for sounds such as sneezes, coughs, or other events that may expel germs from the user's facial orifices. The processor 610 may LEDs 602.

In some embodiments, the visible light camera and/or the thermal camera may be together conjunction to identify surfaces or objects that may be of risk. For example, the visible light camera may identify a table in a restaurant using known computer vision techniques. The thermal camera may then capture a thermal image of the table to see thermal “handprints” on the table from a previous occupant. This may generate an alert to the user and automatically activate the light device. For example, the light device may activate and the smart phone of the user may display a thermal image of the table showing where the surface needs to be cleaned.

The camera may also be used to recognize any other location or object that may indicate a hazard. For example, when the camera captures an image of an area likely to include contaminated individuals, the light device may be automatically activated. For example, the camera(s) may capture an image of a bus, a bus stop, a subway station, a subway train, an airport, an airport runway, a restaurant, an elevator, a hallway, and/or any other type of area that may be occupied by multiple individuals. The camera may use known computer vision techniques to recognize these different areas and automatically activate the light device to provide protection. Thus, the user may walk out of their house, take the subway to work, walk into their office building, and return home at night without needing to manually activate the light device. Instead, the camera and/or thermal camera may recognize dangerous, predefined locations and activate/deactivate the light device automatically as needed.

Some embodiments may include a geo-fencing option that allows the user to automatically designate high-risk areas where the light device should be activated. Some embodiments of the light device may include a GPS chip 630 that provides location information for the device to a microprocessor on the device. Some embodiments may also use a GPS device and a processor on a smart phone to estimate the user's location. Using an app on the smart phone, the user may designate geographic areas in which the light device should be activated or deactivated. For example, the user may “white list” their homes such that the device turns off when they enter their home and turns on his soon as they leave their home. In another example, the user may “black list” areas such as gyms, office buildings, restaurants, and/or other areas that they enter on a regular basis where protection may be needed from contaminants. Combinations of white list and/or black list areas may be used to automatically control activation of the device.

As described above, the light device may physically include an attachment mechanism, such as an adhesive, screws, hook-and-loop strips, and/or the like. The light device may also include one or more memory devices, one or more processors/microprocessors, a communication bus, a power supply and/or a rechargeable power supply, such as a lithium battery. The light device may include controls such as volume, intensity, angle, on/off, and other knobs/buttons for controlling the output, intensity, and direction of the light. The light device may include a charging port and/or a headphone jack, as well as an interface port, such as a USB or micro USB port. The light device may also include a plurality of light-emitting devices, such as LEDs or bulbs. The light device may also include a housing that includes lenses and/or filters that direct the angle/orientation of light emitted from the device.

One or more processors 610 may be communicatively coupled to one or more memory devices 612. The memory devices 612 may store instructions that cause the one or more processors 610 to perform the operations described herein. For example, the memory 612 may store instructions that cause the one or more processors 610 to recognize thermal signatures, recognize voice commands, activate automatically, determine a location using the GPS 630 and compare that location to white/black lists, and so forth.

In some embodiments, the UV light-emitting devices, such as the LEDs 602 may be replaced with light-emitting devices configured to emit light in the visible spectrum. Therefore, all of the features described above may also be used in a device that outputs visible light. These embodiments may be used to illuminate a user's face in the dark when speaking to other people. These embodiments may also be used to light an area directly in front of a user's face for other purposes.

A method of using the device may include receiving an input to activate the device. The input may include any of the triggering inputs described above, such as a user toggling an on-off button, receiving an indication that a thermal image detected a user with an elevated temperature, detecting contaminated surfaces, detecting nearby individuals by voice and/or image, entering/exiting a geo-fenced zone, receiving a command from an app on a smart phone, and so forth. The light device may be affixed to, or embedded in a wearable item of clothing that is worn by the user. The item may include a hat, a scarf, a necklace, a headband, a T-shirt, a jacket, and/or any other item. Receiving the input may cause the light device to activate one or more light emitting devices (e.g., LEDs) to project light in front of, or onto the face of the user.

In the foregoing description, for the purposes of explanation, numerous specific details were set forth in order to provide a thorough understanding of various embodiments. It will be apparent, however, that some embodiments may be practiced without some of these specific details. In other instances, well-known structures and devices are shown in block diagram form.

The foregoing description provides exemplary embodiments only, and is not intended to limit the scope, applicability, or configuration of the disclosure. Rather, the foregoing description of various embodiments will provide an enabling disclosure for implementing at least one embodiment. It should be understood that various changes may be made in the function and arrangement of elements without departing from the spirit and scope of some embodiments as set forth in the appended claims.

Specific details are given in the foregoing description to provide a thorough understanding of the embodiments. However, it will be understood that the embodiments may be practiced without these specific details. For example, circuits, systems, networks, processes, and other components may have been shown as components in block diagram form in order not to obscure the embodiments in unnecessary detail. In other instances, well-known circuits, processes, algorithms, structures, and techniques may have been shown without unnecessary detail in order to avoid obscuring the embodiments.

Also, it is noted that individual embodiments may have been described as a process which is depicted as a flowchart, a flow diagram, a data flow diagram, a structure diagram, or a block diagram. Although a flowchart may have described the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

The term “computer-readable medium” includes, but is not limited to portable or fixed storage devices, optical storage devices, wireless channels and various other mediums capable of storing, containing, or carrying instruction(s) and/or data. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc., may be passed, forwarded, or transmitted via any suitable means including memory sharing, message passing, token passing, network transmission, etc.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

In the foregoing specification, features are described with reference to specific embodiments thereof, but it should be recognized that not all embodiments are limited thereto. Various features and aspects of some embodiments may be used individually or jointly. Further, embodiments can be utilized in any number of environments and applications beyond those described herein without departing from the broader spirit and scope of the specification. The specification and drawings are, accordingly, to be regarded as illustrative rather than restrictive.

Additionally, for the purposes of illustration, methods were described in a particular order. It should be appreciated that in alternate embodiments, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

What is claimed is:

1. A wearable ultraviolet (UV) light device comprising:
one or more UV light-emitting devices; and
a housing that is attachable to a wearable article of clothing, wherein when the housing is attached to the wearable article of clothing, the one or more UV light-emitting devices are positioned and configured to emit light onto or in front of a face of a user.

2. The device of claim 1, further comprising means for attaching the housing to the wearable article of clothing.

3. The device of claim 2, wherein the means for attaching the housing to the wearable article of clothing allow the device to be removable from the wearable article of clothing.

4. The device of claim 1, wherein the one or more light-emitting devices are configured to emit far-UV light.

5. The device of claim 1, wherein the one or more light-emitting devices comprise a plurality of UV light-emitting diodes (LEDs).

6. The device of claim 1, further comprising the wearable article of clothing.

7. The device of claim 6, wherein the wearable article of clothing comprises a hat, and the housing is attached to a bottom side of a brim of the hat.

8. The device of claim 6, wherein the wearable article of clothing comprises a headband or neckband.

9. The device of claim 6, wherein the housing is embedded within the wearable article of clothing.

10. The device of claim 1, further comprising a camera and one or more processors, wherein the camera is configured to capture an image, and the one or more processors are programmed to determine whether to activate the one or more UV light-emitting devices based at least in part on the image.

11. The device of claim 10, wherein the processor is programmed to analyze the image and activate the one or more UV light-emitting devices when the image includes a human approaching the camera.

12. The device of claim 10, wherein the camera comprises a thermal camera, and wherein the processor is programmed to analyze the image and activate the one or more UV light-emitting devices when the image includes a human with a fever temperature.

13. The device of claim 1, further comprising:
a wireless communication chip configured to communicate with a smart phone; and
one or more processors programmed to activate the one or more UV light-emitting devices in response to a command received from the smart phone through the wireless communication chip.

14. The device of claim 1, further comprising:
a motion sensor; and
one or more processors programmed to activate the one or more UV light-emitting devices in response to motion detected by the motion sensor.

15. The device of claim 1, further comprising:
a microphone; and
one or more processors programmed to activate the one or more UV light-emitting devices in response to a voice detected by the microphone.

16. The device of claim 15, wherein the one or more processors are programmed to determine whether the voice detected by the microphone belongs to the user.

17. The device of claim 1, further comprising a power supply and an on/off switch.

18. The device of claim 1, wherein the one or more light-emitting devices are positioned on opposite sides of the face of the user.

19. The device of claim 1, further comprising a global positioning system (GPS) sensor and one or more processors, wherein the one or more processors are programmed to activate the one or more UV light-emitting devices in response to entering a location detected by the GPS sensor.

20. The device of claim 1, further comprising one or more processors programmed to activate the one or more UV light-emitting devices in response to detecting another nearby wearable UV light device.
